(19) United States
(12) Patent Application Publication
(10) Pub. No.: US 2022/0221435 A1
(43) Pub. Date: Jul. 14, 2022

(54) METHOD AND DEVICE FOR IDENTIFYING VOLATILE COMPOUNDS

(71) Applicant: NANOSCENT LTD., Misghav (IL)

(72) Inventors: Yair PASCA, Kiryat Ata (IL); Sima ISRAEL, Haifa (IL); Oren GAVRIELY, Misgav (IL)

(73) Assignee: NANOSCENT LTD., Misghav (IL)

(21) Appl. No.: 17/614,351

(22) PCT Filed: May 27, 2020
(86) PCT No.: PCT/IL2020/050584

§ 371 Date: Nov. 25, 2021

Related U.S. Application Data
(60) Provisional application No. 62/853,054, filed on May 27, 2019.

(51) Int. Cl. G01N 33/00; G06N 20/00

(52) U.S. Cl. CPC G01N 33/0047; G01N 33/0073; G01N 2033/0068; G01N 33/0062; G06N 20/00

(57) ABSTRACT

A volatile compounds (VCs) sensing device is disclosed. The sensing device may include: one or more scent recorders, each scent recorder comprising: a plurality of sensors from which at least two have substantially the same chemical composition and differ in at least one known physical attribute; a controller; and electrodes for connecting the one or more scent recorders to the controller. The at least one known physical attribute may be selected from: the sensor's thickness, the sensor's layer coverage, Layer centering, layer morphology, the sensor's porosity, the sensor's tortuosity, the sensor's particles size, the sensor's particles distribution, thickness uniformity, organic ligands coating to conductive particle, electrode dimensions, gap between electrodes and a water contact angle of the sensors' surface

### FIELD OF THE INVENTION

The present invention generally relates to scent recorders and volatile compounds (VCs) sensing devices. More particularly, the present invention relates to a method and a device for identifying VCs.

### BACKGROUND OF THE INVENTION

Detection of VCs can be done using a variety of devices, also known as, scent recorders, scent readers, artificial noses, VC detectors etc. Some example for such devices may include: chemiresistor sensors, photoionization detectors (PIDs), ultraviolet differential optical absorption spectrometer (UV-DOAS), metal oxide sensors (MOS) and the like. Some of devises are very sensitive to repeatability of the production process, thus may vary in performance between two supposedly identical devises. For example, physical attributes, such as thickness, width, density, distribution of particles and the like. One type of such a device, the chemiresistor sensor is known to be sensitive to diversity the physical attributes of the sensor.

Chemiresistor sensors are sensors that can detect the presence of volatile compound (VCs). A chemiresistor sensor includes a material or structure that changes its electrical resistance in response to changes in the nearby chemical environment, for example, due to the presence of VCs. Commercial chemiresistor sensors for sensing VCs include a sensing element made from one of: carbon nanotubes, graphene, carbon nanoparticles and the like. Another optional sensor includes a sensing element made form metallic nanoparticles cores coated with organic ligands. The organic ligands are bonded to the surface of the metallic core at one end and are configured to be weakly bonded (e.g., interact) to a VC at the other end. The most suitable and widely used cores are nanoparticles of: Au, Pt, Pd Ag and further also alloys consisting of Ni, Co, Cu, Al, Au/Ag, Au/Cu, Au/Ag/Cu, Au/Pt, Au/Pd, Au/Ag/Cu/Pd, Pt/Rh, Ni/Co, and Pt/Ni/Fe.

The most common type of organic ligands that can bond with the surface of a metallic particle having one of the above listed metallic cores is thiol (sulfides). Thiols can be bonded with the metallic cores via functional groups such as: alkylthiols with C3-C24 chains, ω-functionalized alkanethiolates, arenethiolate, (γ-mercaptopropyl) tri-methyloxysilane, dialkyl disulfides, xanthates, oligonucleotides, polynucleotides, peptides, proteins, enzymes, polysaccharides, and phospholipids.

Each particle in a chemiresistor sensor may interact with more than one types of VC. However, the specific chemical—physical nature of each particle, in particular the functional groups in the organic ligands makes each of them capable of forming larger number of weak bonds with a major (e.g., favorable) type of VC. However, other VCs may also be weakly bonded to the ligands. The VC may interact or absorb in several sites of the nanoparticle, for example, core surface, bonding group, the functional groups and the like. Sensing elements known in the art usually includes a single type of particle. In such case each sensing element may be sensitive to or detect a single type of VC or may detect two or more types of VC, but without the ability to simultaneously and in real time separate between the two types of particles.

Chemiresistor sensors may be printed using an ink-jet printer or may be deposited using any other known method. The deposited “ink” may include one or more types of particles. Usually more than one sensor is printed to form a single scent recorder. These production methods may yield non-identical sensors within one scent recorder. For example, the sensors although having substantially the same chemical composition may vary in the thickness, width, length, distribution of particles or in any other physical attribute due to, for example, inaccuracy in the printing process. These differences affect the performance of each sensor. Some examples for such performance, may include decline or increase in measurement attributes, such as, the base resistance, base conductivity, electrical noise, base current and based voltage. Such measurement attributes may be affected from differences in the thickness, coverage, number of deposited layers of the sensors and the like.

Accordingly, there is a need for a method (e.g., computerized method) that can overcome the none-repeatability of the sensors and even take advantage of the none-repeatability.

### SUMMARY OF THE INVENTION

Some aspects of the invention may be related to volatile compounds (VCs) sensing device, comprising: one or more scent recorders, each scent recorder comprising: a plurality of sensors from which at least two have substantially the same chemical composition and differ in at least one known physical attribute; a controller; and electrodes for connecting the one or more scent recorders to the controller. In some embodiments, the at least one known physical attribute may be selected from: the sensor's thickness, the sensor's layer coverage, Layer centering, layer morphology, the sensor's porosity, the sensor's tortuosity, the sensor's particles size, the sensor's particles distribution, thickness uniformity, organic ligands coating to conductive particle, electrode dimensions, gap between electrodes and a water contact angle of the sensors' surface. In some embodiments, each sensor may be composed from a plurality of conductive particles each being covered by organic ligands.

In some embodiments, the controller may be configured to: receive measured attribute values from of the at least two sensors; receive signals from the at least two sensors, in response to exposure to at least one type of VC; extract values of a feature from the signals; and find mathematical correlation between the extracted values and a corresponding measured attribute value. In some embodiments, the received signals are from at least one known VC and the controller may further be configured to associate the mathematical correlation with the at least one known VC and store in a database. In some embodiments, the received signals are from at least one unknown VC and the controller may further be configured to: identify the at least one unknown VC based on the stored mathematical correlation. In some embodiments, identifying the at least one unknown VC comprises at least one of: identifying the type of the VC and the concentration of the VC.

In some embodiments, the measured attribute values are selected from: base resistance (e.g., background resistance), base conductivity, electrical noise, base current, based voltage, base frequency and base amplitude.

In some embodiments, the extracted feature values are selected from: the maximal subtracted resistance, the difference between maximum and minimum values, the average value, the maximum value, the minimum value, the first time derivative, the second time derivative, signal to noise ratio, incline gradient, decline gradient, rise time, overshooting value relative to steady state value, oscillation decay in time and oscillation frequency.

In some embodiments, the mathematical correlation is one of: a linear correlation, a parabolic correlation, a polynomial correlation, logarithmic correlation, exponential correlation and power correlation.

Some additional aspects of the invention may be directed to method of finding mathematical correlation between extracted feature values and corresponding measured attribute values, comprising: receiving at least one measured attribute from at least two sensors having substantially the same chemical composition and differ in at least one physical attribute; receiving signals from the at least two sensors, in response to exposure to at least one VC; extracting values of a feature from at least some of the plurality of signals; finding mathematical correlation between the extracted values and a corresponding measured attribute values.

In some embodiments, the exposure is to at least one known and the method may further include associating the mathematical correlation with the at least one known VC and store in a database.

In some embodiments, the method may further include: receiving signals from the at least two sensors, in response to exposure to at least one unknown VC; and identifying the at least one unknown VC based on the stored mathematical correlation.

In some embodiments, the method may further include training a machine learning (ML) module to identify the known VC based on the mathematical correlation. In some embodiments, the method may further include: receiving signals from the at least two sensors, in response to exposure to at least one unknown VC; and identifying the at least one unknown VC based on the stored mathematical correlation, using the trained ML module.

In some embodiments, the measured attribute value may be selected from: base resistance (e.g., background resistance), base conductivity, electrical noise, base current, based voltage, base frequency and base amplitude. In some embodiments, the extracted feature values are selected from: the maximal subtracted resistance, the difference between maximum and minimum values, the average value, the maximum value, the minimum value, the first time derivative, the second time derivative, Signal to noise ratio, incline gradient, decline gradient, rise time, overshooting value relative to steady state value, oscillation decay in time and oscillation frequency. In some embodiments, the mathematical correlation is one of: a linear correlation, a parabolic correlation, a polynomial correlation, logarithmic correlation, exponential correlation, and power correlation.

In some embodiments, the at least one physical attribute is selected from: the sensor's thickness, the sensor's layer coverage, the sensor's porosity, the sensor's tortuosity layer centering, layer morphology, the sensor's particles size, the sensor's particles distribution, thickness uniformity, organic ligands coating to conductive particle, electrode dimensions, gap between electrodes and a water contact angle of the sensors' surface.

In some embodiments, each sensor may be composed from a plurality of conductive particles each being covered by organic ligands.

Some additional aspects of the invention may be directed to a method of calibrating sensors included in a scent recorder. In some embodiments, the method may include: receiving at least one measured attribute from a plurality of sensors included in the scent recorder from which at least two sensors have substantially the same chemical composition and vary in at least one physical attribute, and wherein values of the at least one physical attribute are unknown; receiving signals from the at least two sensors, in response to exposure to at least one type of VC; extracting values of a feature from at least some of the signals; finding mathematical correlation between the extracted values and a corresponding measured attribute values; and calibrating values extracted from new signals received from the at least two sensors using the mathematical correlation.

In some embodiments, the measured attribute values are selected from: base resistance, base conductivity, electrical noise, base current, based voltage, base frequency and base amplitude. In some embodiments, the extracted feature values are selected from: the maximal subtracted resistance, the difference between maximum and minimum values, the average value, the maximum value, the minimum value, the first time derivative, the second time derivative, Signal to noise ratio, incline gradient, decline gradient, rise time, overshooting value relative to steady state value, oscillation decay in time and oscillation frequency.

In some embodiments, the mathematical correlation is one of: a linear correlation, a parabolic correlation, a polynomial correlation, logarithmic correlation, exponential correlation and power correlation. In some embodiments, the at least one physical attribute is selected from: the sensor's thickness, the sensor's layer coverage, the sensor's porosity, the sensor's tortuosity layer centering, layer morphology, the sensor's particles size, the sensor's particles distribution, thickness uniformity, organic ligands coating to conductive particle, electrode dimensions, gap between electrodes and a water contact angle of the sensors' surface. In some embodiments, each sensor is composed from a plurality of conductive particles each being covered by organic ligands.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A, is an illustrations of scent recorder according to some embodiments of the invention;

FIG. 1B is an illustration of a chemiresistor sensor according to some embodiments of the invention;

FIG. 1C is an illustration of a particle in a chemiresistor sensor according to some embodiments of the invention;

FIG. 2 is a block diagram of a VC sensing device according to some embodiments of the invention;

FIG. 3 is a flowchart of a method of finding mathematical correlation between extracted value and a measured attribute value according to some embodiments of the invention;

FIG. 4A is a flowchart of a method of identifying a VC according to some embodiments of the invention;

FIG. 4B is a flowchart of a method of calibrating a scent recorder according to some embodiments of the invention;

FIG. 5 presents a graph showing the relationship between a physical attribute (the number of deposited layers) and a measured attribute value (the base resistance), and some micrographs of sensors having different numbers of deposited layers, according to some embodiments of the invention;

FIG. 6 presents a graph showing the relationship between another physical attribute (the coverage (e.g., area) of the deposited layers) and a measured attribute value (the base resistance), and some micrographs of sensors having different covered areas, according to some embodiments of the invention;

FIG. 7 presents a graph showing an extracted feature, according to some embodiments of the invention;

FIG. 8 presents a graph showing linear correlation between an extracted feature and measured attribute value according to some embodiments of the invention;

FIG. 9 presents a graph showing power correlation between another extracted feature and measured attribute value according to some embodiments of the invention;

FIGS. 10A-10D presents graphs demonstrating the various steps included in the method of FIG. 4C according to some embodiments of the invention;

FIG. 11 presents graph showing an influence of accelerated degradation in the physical attributes of a sensor on the calculated mathematical correlation, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF EMBODIMENTS OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components, modules, units and/or circuits have not been described in detail so as not to obscure the invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, “synchronizing” or the like, may refer to operation(s) and/or process(es) of a computer, a computing platform, a computing system, or other electronic computing device (e.g., a wearable electronic computing device), that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

As used herein the term “volatile compound” (VC) may be related to any volatile material, either organic or inorganic compound. Some examples for VCs may include: Cr₂, O₂, H₂, CH₄, NH₃, CH₃OH, SOₓ, NOₓ BTEX, Benzene, Toluene, Ortho-Xylene, Para-Xylene, Meta-Xylene and Ethylbenzene and the like.

The term “chemical composition” of a sensor, in the context of this application, refers to the chemical formula(s) of compound. Accordingly, two sensors having substantially the same chemical composition may include having similar components having the same chemical formulas. For example, sensors having substantially the same chemical composition may include sensors having the same conductive cores (e.g., same alloy, same conductive oxide coating and the like) covered by the same type of organic ligands.

As used herein, the term “physical attribute” of a sensor refers to any physical property of a sensor that does not affect the chemical composition of each of the components/particles included in a sensor. For example, physical attribute may be at least one of: the sensor's thickness, the number of deposited sensing layers, the sensor's layer coverage, sensor's layer centering, sensor's layer morphology, the sensor's particles size, the sensor's particles distribution, sensor's layer thickness uniformity, organic ligands coating to conductive particle (e.g., a metal, conductive oxide, graphite, etc.) ratio, electrode dimensions, gap between electrodes, a water contact angle of the sensors' surface, deposited layer porosity, deposited layer tortuosity and the like.

As used herein, the term “measured attribute value” of a scent recorder refers to any measured property that can be measured by a scent recorder that was either controllably exposed to a specific VC (e.g., a calibration gas) or not exposed to any VC. In such case, the scent recorder may measure the base/background concentrations of VC in the air. For example, a measured attribute value may be or may include at least one of: base resistance (e.g., background resistance), base conductivity, electrical noise, base current, based voltage, base frequency, base amplitude, and the like. In some embodiments, the base resistance may be measured at at least one of the following conditions: under pure gas such as N₂, in indoor or outdoor air, during flow of any of the gasses, at room temperature or at any other temperature of interest, at room pressure or at any other pressure of interest, under vacuum and the like.

The term “extracted feature values”, as used herein, refers to any mathematical property/attribute that may be derived from measured values from a signal received from a sensor. For example, extracted feature values may include one or more of: the difference between maximum and minimum values, the average value, the maximum value, the minimum value, the first time derivative, the second time derivative, signal to noise ratio, incline gradient, decline gradient, rise time, overshooting value relative to steady state value, oscillation decay in time, oscillation frequency and the like.

As used herein, the term “mathematical correlation” may include any correlation between two values (e.g., numbers). for example, mathematical correlation may be selected from: a linear correlation, a parabolic correlation, a polynomial correlation, logarithmic correlation, exponential correlation, and power correlation. Mathematical correlation according to some embodiments of the invention may include correlations between the extracted feature values and a measured attribute values, for example, the measured maximum value of the signal and the signal noise.

Aspects of the invention may be directed to provide more reliable, stable VC measurements that are not sensitive to: chemical degradation, drift (e.g., accumulation of VCs), wear and fatigue processes within VC sensors or scent recorders. A system and method according to some embodiments of the invention may include finding mathematical correlation(s) between values measured by the sensors or scent recorders. These mathematical correlations may not be sensitive to degradation processes of the sensors over time. These degradation processes may cause a drift in values measured by the sensors. In some embodiments, the degradation processes may result in variations in at least some of the physical attributes between two sensors included in a single scent recorder.

Some aspects of the invention may be related to deliberately causing differences in the physical attributes between two or more sensors included in a single scent recorder in order to determine the mathematical correlation(s) for the scent recorder. In some embodiments, the mathematical correlation may correspond to the type of physical attribute. For example, for sensors having the same chemical composition (e.g., the same conductive cores covered by the same type of organic ligands) that may be printed in various thicknesses, the mathematical correlation between the average measured value and the base resistance may be the same regardless of the sensor's thicknesses. Meaning that any change in a physical attribute (either as part of a degradation process over time or deliberately conducted) may result in corresponding changes in both the measured attribute values and extracted feature values, but the correlation between the two may stay the same, as presented in the nonlimiting example given with respect to FIG. 11.

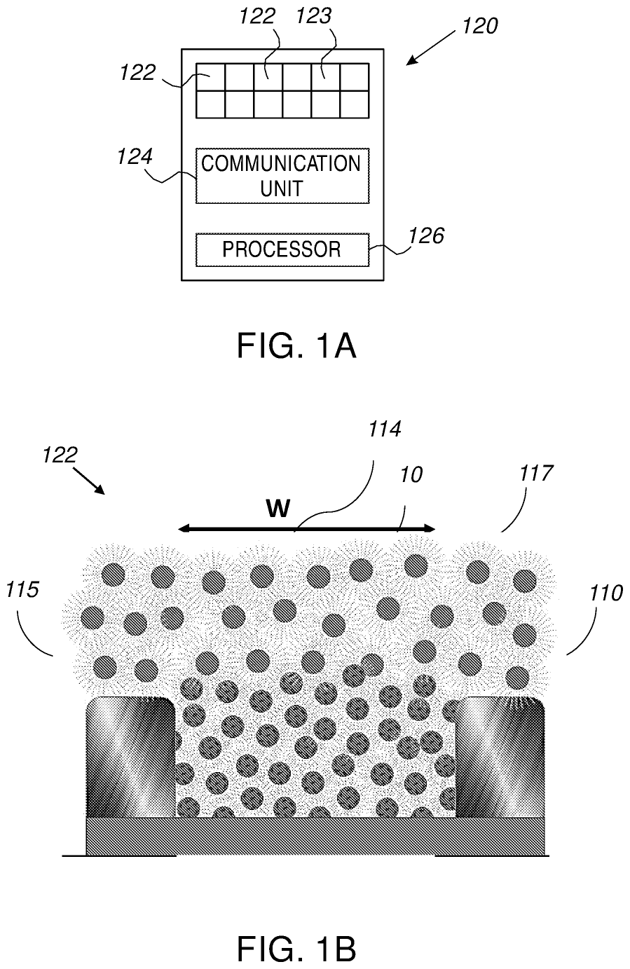

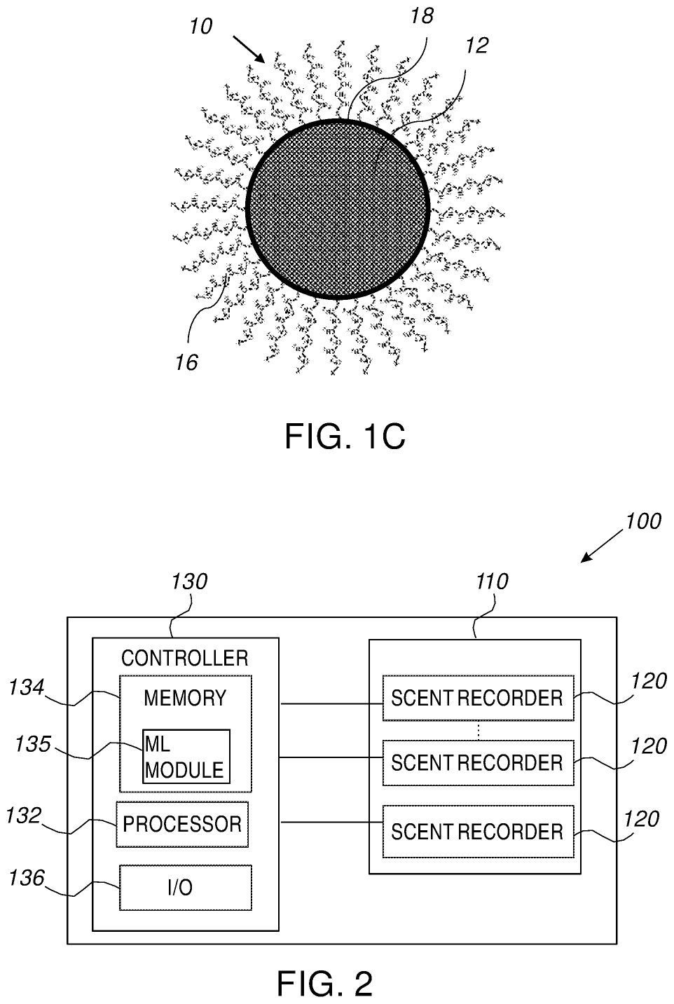

Reference is now made to FIG. 1A which is an illustration of a scent recorder 120 according to some embodiments of the invention. Scent recorder 120 may include one or more sensors 122 and 123, a communication unit (e.g., wireless or wired unit) 124 for communicating with a controller 130 (illustrated in FIG. 2) and a processor 126. Processor 126 (e.g., a chip) may collect readings from one or more sensors 122 and 123 and send them as a signal to controller 130 via communication unit 124.

Sensors 122 may be any sensor that may detect the presence of one or more volatile compounds (VCs). For example, sensor 122 may include a material or structure that changes its electrical resistance in response to changes in the nearby chemical environment, for example, due to the presence of VCs. In some embodiments, sensor 122 may be a chemiresistor sensor for sensing VCs that may include a sensing element made from one of: carbon nanotubes, graphene, carbon nanoparticles, conductive polymers and the like. These chemiresistor sensors are sensitive to cleaning and regeneration cycles which are required after each measurement, due to the nonuniform nature of the sensor's material. Another optional chemiresistor sensor 122 may include conductive nanoparticles cores coated with organic ligands, as illustrated and discussed with respect to FIGS. 1B and 1C. In some embodiments, at least two sensors 122 may have substantially the same chemical composition and may differ in at least one physical attribute. In some embodiments, one or more sensors 123 may have a different chemical composition than sensors 122. It should be appreciated that scent recorder 120 may include an array of sensors all having the same chemical composition, such as sensors 122 or may include an array with two or more types of sensors, e.g., having different chemical composition. According to some embodiments, scent recorder 120 may include at least two sensors from each type of sensors in the array of sensors 122, 123.

Reference is now made to FIG. 1B which is an illustration of a chemiresistor sensor according to some embodiments of the invention. A chemiresistor sensor 122 may include: two electrodes 110 and 115 and a sensing element 114. Electrodes 110 and 115 may be electrically connected to electrical connectors (not insulated). Sensing element 114 may include a plurality of particles 10 (illustrated and discussed with respect to FIG. 1C). Particles 10 may be deposited between or on electrodes 110 and 115, using for example, ink jet printing. In some embodiments, the thickness H of sensing element 114 may vary between at least some of deposited sensors 122 included in scent recorder 120. In some embodiments, the width W or the length (not illustrated) may vary between at least some of deposited sensors 122 included in scent recorder 120. In some embodiments, the distribution or density of particles 10 may vary between at least some of deposited sensors 122 included in scent recorder 120. For example, some of sensors 122 may include sensing elements 114 having a first density or a second density or a combination the first and second densities, as illustrated. Any other number of different densities may appear in different sensors 122.

Reference is now made to FIG. 1C which is an illustration of a particle for a chemiresistor sensor according to some embodiments of the invention. In some embodiments, particle 10 may include a nanoparticle core 12. Nanoparticle core 12 may be made of a conductive material selected from a group consisting of: Jr, Jr-alloy, IrOₓ, Ru, Ru-alloy, RuOₓ Au, Pt, Pd, Ag, Ni, Co, Cu, Al, Au/Ag, Au/Cu, Au/Ag/Cu, Au/Pt, Au/Pd, Au/Ag/Cu/Pd, Pt/Rh, Ni/Co, Pt/Ni/Fe and any combination thereof. In some embodiments, nanoparticle core 12 may be made from any conductive metallic oxide. In some embodiments, the average diameter of nanoparticle core 12 may be of at most 100 nm, for example, 50 nm, 20 nm, 10 nm, 5 nm and 1 nm.

In some embodiments, nanoparticle core 12 may include a single metal or alloy and may have crystalline structure. In some embodiments, nanoparticle core 12 may include a single metal or alloy and may have an amorphous structure. In some embodiments, nanoparticle core 12 may include more than one metal or alloy, for example, a conductive oxide such as RuOₓ, IrOₓ and a metal, such as, Ru alloy, Ru, Jr alloy, Ir.

In some embodiments, particle 10 may further include a plurality of organic ligands 16 bonded, for example, via covalent bonds, from one side to the metallic core and capable of interacting with a VC. In some embodiments, the organic ligands are selected from a group consisting of: thiols, diazoniums, silanes, carboxylic acids, tri-chloro, methoxy, ethoxy, tri hydroxide, di-chloro, chloro and the like. In some embodiments, more than one type of organic ligand may be bonded with one of core 10. For example, a core 10 that includes crystalline RuOₓ may be coated with organic ligands 16 of methoxy silane and trichloro silane. In some embodiments, the organic ligands can bond from one side, the side that includes the bonding group, for example, the chloro-silane, group with the surface of core 10 and from the other side with a VC, for example, a specific functional group can be added to the ligand chain that is configured to target (e.g., interact with) a specific VC. In some embodiments, the VC may interact with the branch of the organic ligands, for example, via VDW bonds, hydrogen bonds, Pi-Pi orbital interaction (for aromatics), dipole-dipole interaction (for polar), steric interference, charge to charge interaction, coordination bond, etc.

In some embodiments, nanoparticle core 10 may at least be partially covered with a conductive oxide layer 18 including at least one of: IrOₓ and RuOₓ or any other conductive oxides. In some embodiments, when nanoparticle cores 10 include crystalline or amorphous Jr, Jr-alloy, Ru or Ru-alloy, a thin oxidation layer may form on at least a portion of the surface of nanoparticle core 10. The thin oxidation layer may be formed due to the exposure of the nanoparticle cores to air or oxygen. The thickness of the oxidation layer may be of few nanometers, for example, a native oxide layer of 1-2 nm. In some embodiments, such an oxidation layer may allow stronger bonding between organic ligand 16 and the surface of cores 10.

Reference is now made to FIG. 2 which is a block diagram of a VC sensing device according to some embodiments of the invention. A device 100 may include one or more scent recorders 120, for example, an array 110 of at least 2 scent recorders 120. In some embodiments, devise 100 may further include a controller 130.

In some embodiments, controller 130 may include a processor 132, a memory 134 and an input/output unit 136. Processor 132 may be a central processing unit (CPU) processor, a chip, PCB or any suitable computing or computational device. Memory 134 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 134 may be or may include a plurality of, possibly different memory units. Memory 134 may be a computer or processor non-transitory readable medium, or a computer non-transitory storage medium, e.g., a RAM. In one embodiment, a non-transitory storage medium such as memory 134, a hard disk drive, another storage device, etc. may store instructions or code which when executed by a processor may cause the processor to carry out methods as described herein.

In some embodiments, memory 134 may store an operation system that may include any code segment designed and/or configured to perform tasks involving coordination, scheduling, arbitration, supervising, controlling or otherwise managing operation of processor 132. In some embodiments, memory 134 may store a code or instructions for identifying a VC and or calibrating a scent recorder such a scent recorder 120.

In some embodiments, memory may store a Machine Learning (ML) module 135. ML module 135 may, for example, use any appropriate type of a neural network (NN) based ML model for identifying VCs. ML module 134 may be trained to identify VCs.

Input/output unit 136 may include any unit/units that allows controller 130 to send and/or receive information from external devices, for example, scent recorders 120, 120A and 120B and user devices (e.g., laptops, tables, smartphones and the like). Input/output unit 136 may be or may include any suitable input devices, components or systems, e.g., a detachable keyboard or keypad, a mouse and the like. Input/output unit 136 may include one or more (possibly detachable) displays or monitors, speakers and/or any other suitable output devices. Any applicable input/output (I/O) devices may be connected to controller 130, for example, a wired or wireless network interface card (NIC), a universal serial bus (USB) device or external hard drive.

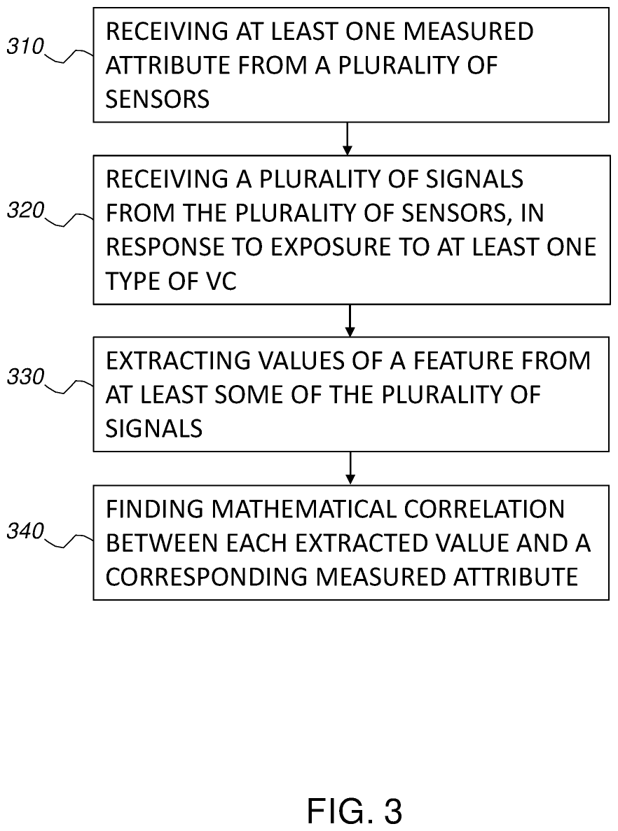

Reference is now made to FIG. 3 which is a flowchart of a method of finding mathematical correlations between set of extracted feature values and the corresponding measured attribute values according to some embodiments of the invention. The method of FIG. 3 may be conducted by processor 126 or controller 130 or by any other suitable computing device.

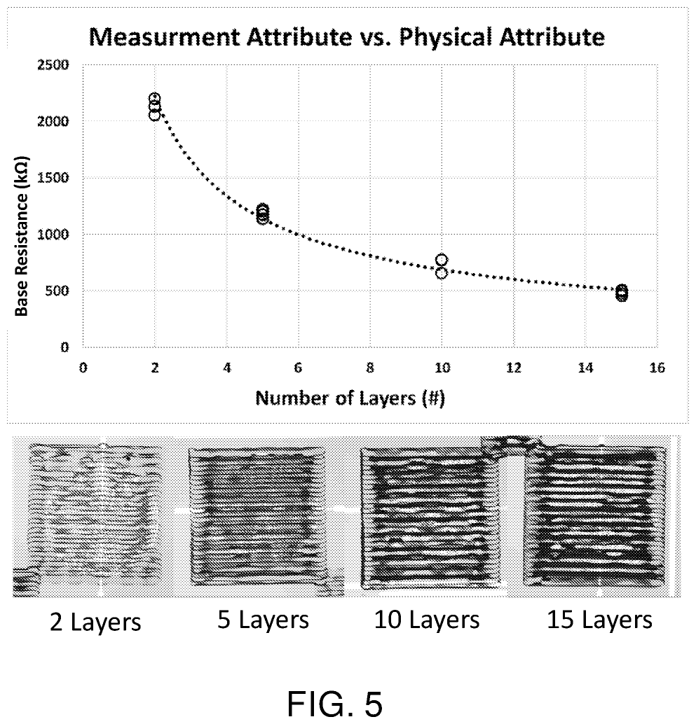

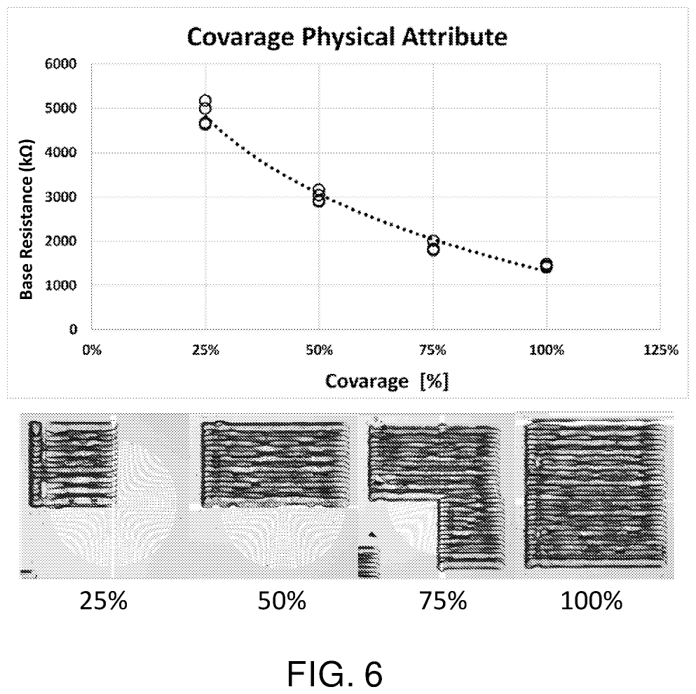

In step 310, at least one measured attribute value may be received from a plurality of sensors included in the scent recorder (e.g., sensors 122 and 123). In some embodiments, at least two sensors (e.g., sensors 122) may have substantially the same chemical composition and may vary in at least one physical attribute. For example, two or more sensors 122 included in a single scent recorder 120 may differ in at least one of: the sensor's thickness, the number of deposited layers, the sensor's layer coverage (e.g., how much electrode area is covered by the sensor), layer centering, layer morphology (e.g., porosity level, tortuosity level, etc.) the sensor's particles size, the sensor's particles distribution, the sensor's thickness uniformity, ratio between organic ligands coating to conductive particle, electrode dimensions, gap between electrodes and a water contact angle of the sensors' surface (e.g., hydrophilicity or hydrophobicity). Two nonlimiting examples for the connection between measured attribute values and physical attributes are given in FIGS. 5 and 6.

In some embodiments, the difference in the physical attribute may be a result of variation in the scent recorder production process. In such case similar variations in the physical attribute may be included in all sensors 122 included in scent recorders 120 fabricated in the same fabrication line. For example, all scent recorders 120 fabricated in the same fabrication line may each include the same number of sensors 122 that may vary from one another at least in the particles' distribution. In some embodiments, the difference is physical attribute may be a result of degradation over time (e.g., due to wear and fatigue processes) of various sensors 122 in scent recorder 120. In some embodiments, the difference in physical attributes may be deliberately caused during the production process. For example, scent recorders 120 may be fabricated with two or more of sensors 122 having different thicknesses.

In some embodiments, the received measured attribute values may be selected from: base resistance (e.g., background resistance) base conductivity, electrical noise, base current, based voltage, base frequency and base amplitude. In some embodiments, the measured attribute values may be measured and recorded (e.g., in memory 134) in one of the following conditions: under pure gas such as N₂, under indoor or outdoor air, during flow of any of type of known gasses, at room temperature or at any other temperature of interest, at room pressure or at any other pressure of interest, under vacuum and the like.

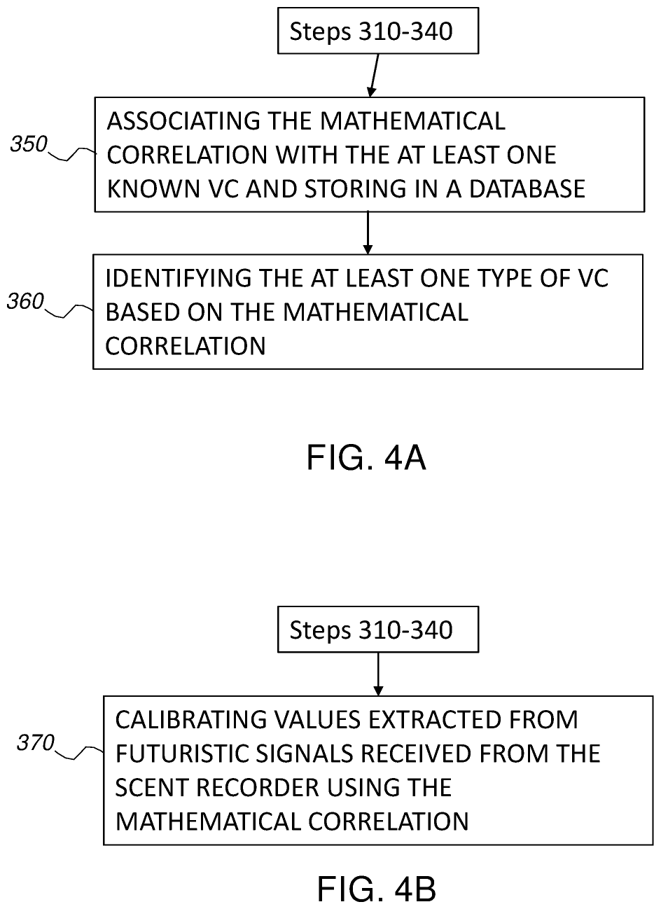

In step 320, signals may be received from the of two or more sensors, in response to exposure to at least one type of VC. The exposure may change the resistance of each sensor. In some embodiments, the at least one type of VC may be a known VC, for example, the type (e.g., chemical composition) and/or the concentration of the VC may be known. Such known VCs may be used to associate the mathematical correlation with the at least one known VC and store the association in a database (e.g., memory 134). In some embodiments the VCs may be a unknown VCs and system 100 may be configured to identify the unknown VC, as discussed with respect to method of FIG. 4A.

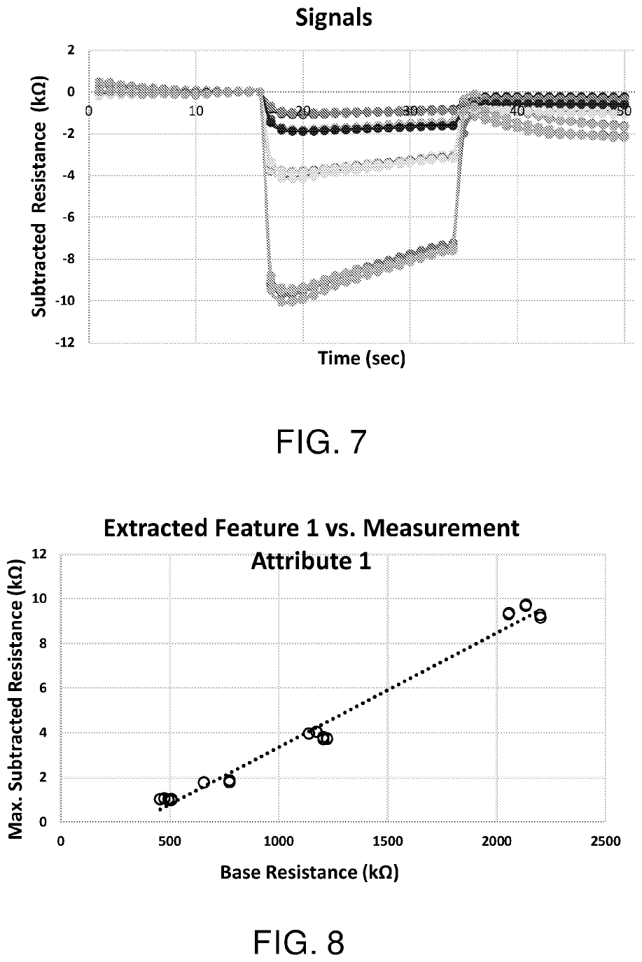

In step 330, values of a feature may be extracted from at least some of the signals. The extracted values may be selected from: maximal subtracted values (e.g., the difference between the maximal resistance and the base resistance), the difference between maximum and minimum values, the average value, the maximum value, the minimum value, the first time derivative, the second time derivative, signal to noise ratio, incline gradient, decline gradient, rise time, overshooting value relative to steady state value, oscillation decay in time and oscillation frequency. A nonlimiting example, for an extracted feature value as a function of time is given in FIG. 7.

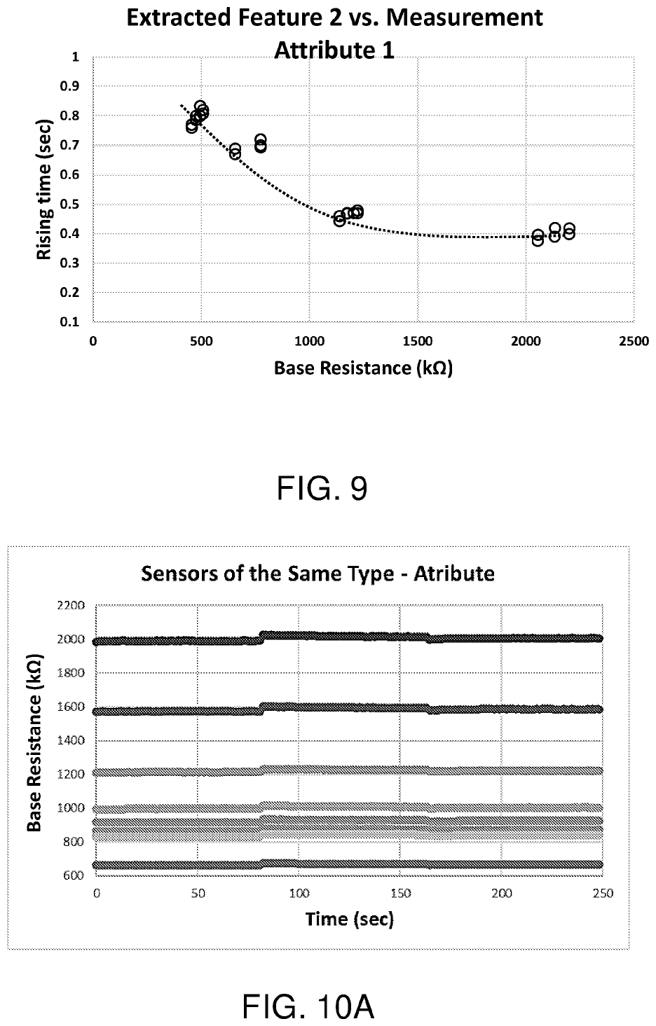

In step 340, mathematical correlation may be found between the extracted values and a corresponding measured attribute values. In some embodiments, the mathematical correlation may be selected from a linear correlation, a parabolic correlation, a polynomial correlation, logarithmic correlation, exponential correlation, power correlation and the like. For example, a polynomial correlation may be found between the base resistance and the first time-derivative of the signal. Some nonlimiting examples for mathematical correlations are given herein below with respect to FIGS. 8 and 9.

Reference is now made to FIG. 4A which is a flowchart of a method of identifying volatile compounds (VCs) according to some embodiments of the invention. In some embodiments, the method of FIG. 4A may include steps 310-340 of the method of FIG. 3. In some embodiments, the scent recorders 120 may first be exposed to at least one known VC. For example, a scent recorder 120 having a known type of variation in at least one physical attribute (e.g., thickness) between at least two sensors 122 included in scent recorder 120 may be exposed to a variety of known types and/or known concentrations of VCs. In some embodiments, mathematical correlations may be found or calculated (e.g., by processor 123 or controller 130) for each type/concentration of VC. The calculated correlation may associate with the at least one known VC and store in a database (e.g., in a lookup table), in step 350. In some embodiments, the correlation may be used to train an ML module. The ML module may receive signals from a plurality of known VCs and the corresponding calculated correlations. The ML module may identify and store (e.g., in memory 134) a typical calculated correlation for each type of known VCs. The typical calculated correlation may be used to later identify unknown types of VC.

In some embodiments, the stored correlations may be used to identify unknown VCs. In some embodiment, the same scent recorders 120 or scent recorders 120 made in the same production line having the same known type of variation in at least one physical attribute may be used. Scent recorder 120 may be exposed to one or more unknown VCs. In some embodiments, the method may include steps 310-340 of the method of FIG. 3. In step 360, at least one type of VC may be identified based on the stored mathematical correlation. In some embodiments the identification may be done using the trained ML module (e.g., module 135). For example, controller 130 may be configured to use ML module 135 to identify at least the type and/or concentration of the one or more unknown VCs.

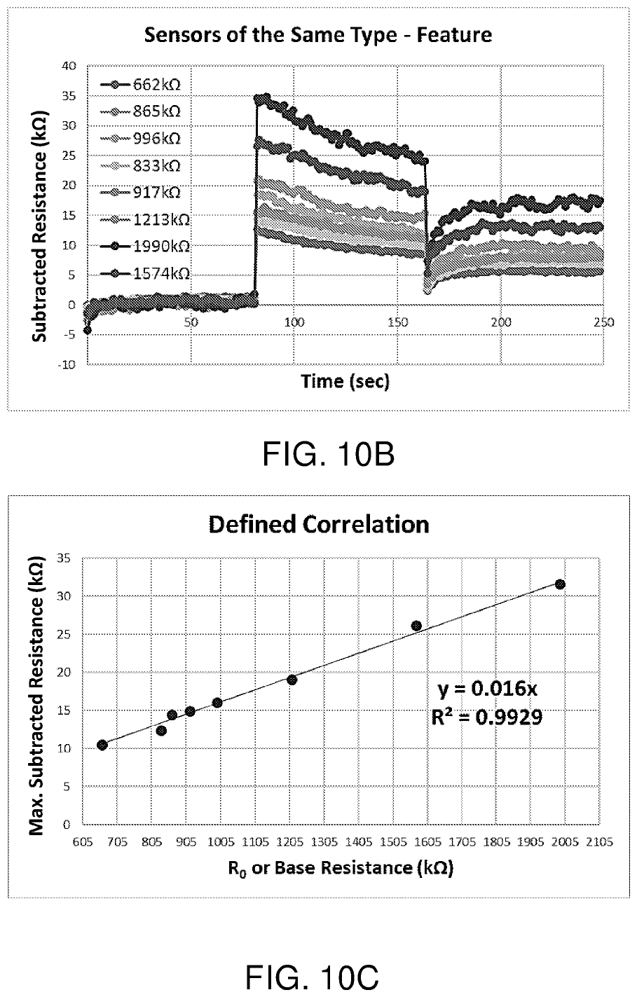

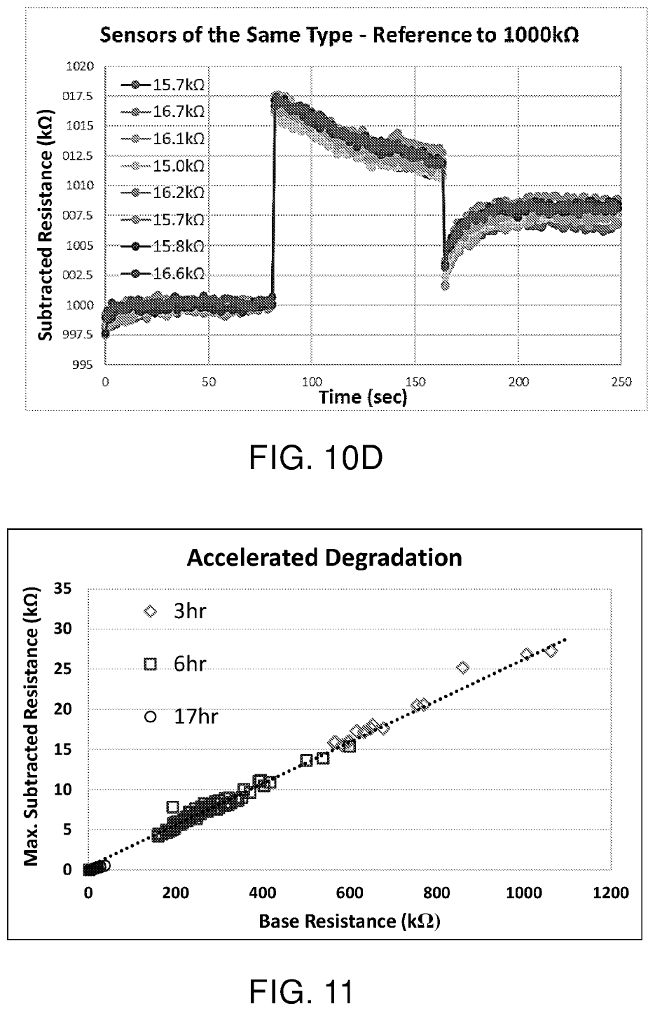

Reference is now made to FIG. 4B which is a flowchart of a method of calibrating a scent recorder according to some embodiments of the invention. In some embodiments, scent recorder 120 may include two or more sensors 122 having unknown values of the at least one physical attribute. Embodiments of the method may include steps 310-340 of the method of FIG. 3. In some embodiments, the at least one unknown physical attribute may result from variation during the production process or from wear processes during the scent recorder service time. In step 360, the mathematical correlation may be used to calibrate values extracted from futuristic signals received from the scent recorder. Some nonlimiting examples of such a calibration process are presented in FIGS. 10A-10D.

Reference is now made to FIG. 5 which presents a graph showing a nonlimiting example for relationship between a physical attribute (the number of deposited layers) and measured attribute values (the base resistance) taken form 16 sensors, and some micrographs of sensors having different numbers of deposited layers, according to some embodiments of the invention. As shown in the graph of FIG. 5 there is a mathematical correlation between the number of deposited layers and the base resistance of each sensor.

Reference is now made to FIG. 6 presents a graph showing a nonlimiting example for relationship between another physical attribute, the coverage (e.g., area) of the deposited layers, having the same number of deposited layers, and measured attribute values (the base resistance) taken form 16 sensors, and some micrographs of sensors having different covered areas, according to some embodiments of the invention. As shown in the graph of FIG. 6 there is a mathematical correlation between the area covered by the deposited layers and the base resistance of a sensor.

The calculated correlations shown in FIGS. 5 and/or 6 may be applicable to all sensors having the same chemistry, produced in the same production line under the same production conditions.

Therefore, since measuring the measured attribute values is a lot simpler than measuring the physical attribute, changes in measured attribute values, such as the base resistance, may be indicative to changes in one of more physical attribute.

Reference is now made to FIG. 7 which shows graphs of extracted feature values from various sensors, according to some embodiments of the invention. The graphs show subtracted resistance values (the measured resistance subtracted by the base resistances) measured over time in 16 sensors having substantially the same chemical compositions. The sensors differ in the value of at least one physical attribute (e.g., the thickness of the sensor). The drop in the subtracted resistances in all sensors indicates the exposure of the sensor to a VC. The exposure was to a known VC at a known concentration.

Reference is now made to FIG. 8 which presents a graph showing linear mathematical correlation between extracted feature values and measured attribute values according to some embodiments of the invention. The graph of FIG. 8 shows linear correlation between the maximal subtracted resistance values (e.g., the difference between the maximal resistance and base resistance) extracted feature and the base resistance measurement attribute. The correlation was based on 16 sensors with different sensing layer thickness.

Reference is now made to FIG. 9 which presents a graph showing power mathematical correlation between another extracted feature values and measured attribute values according to some embodiments of the invention. The graph of FIG. 9 shows polynomial correlation between rising time extracted feature and base resistance measurement attribute. The correlation is based on 16 sensors with different sensing layer thickness.

Reference is now made to FIGS. 10A, 10B, 10C and 10D which present graphs demonstrating the various steps included in the calibration method of FIG. 4C according to some embodiments of the invention.

The graphs in FIG. 10A show the base resistances measured over time in 8 sensors which differ only in the sensor thickness. The base resistances of FIG. 10A is a nonlimiting example, for the measured attribute values, received in step 310 of FIG. 3. As shown hereinabove, the difference in the base resistance may be indicative of changes in the thickness between the different sensors.

The graphs in FIG. 10B show the feature subtracted resistances (the difference between the measured resistance and the base resistance) value extracted from signals received from the 8 sensors over exposure cycle (a nonlimiting example for step 320). The rise in the leveled resistances value in all sensors indicates the exposure of the sensor to a VC. The exposure was to a know VC at a known concentration.

An additional extracted feature may be calculated from the graphs of FIG. 10B. The maximal subtracted resistance value from each signal may further be calculate (e.g., as in step 330 of the method of FIG. 3).

| | | | Maximal Subtracted |
|---|---|---|---|
| | | | resistance = max |
| | Maximal resistance | Base resistance | resistance − base |
| | [kΩ] at | [kΩ] at | resistance |
| Sensor # | t = 82 seconds | t = 81 seconds | [kΩ] |
| 1 | 661.9598 | 662 | 10.42377 |
| 2 | 864.6587 | 865 | 14.44472 |
| 3 | 994.5364 | 996 | 16.05755 |
| 4 | 832.6055 | 833 | 12.54964 |
| 5 | 915.9909 | 917 | 14.87218 |
| 6 | 1210.547 | 1213 | 19.06258 |
| 7 | 1986.338 | 1990.5 | 31.5549 |
| 8 | 1572.592 | 1574 | 26.13079 |

A nonlimiting example for step 340 of FIG. 3 is given in FIG. 10C. The graph in FIG. 10C shows the linear correlation between the extracted maximal subtracted resistance value as function of the base resistance.

The graph in FIG. 10D shows a nonlimiting example, for step 670 of FIG. 4C, calibrating values extracted from a new signal received from the scent recorder using the mathematical correlation calculated in FIG. 10C. As shown in FIG. 10D after calibration all the received signals presents similar resistance behavior over exposure cycle, regardless of the differences in the physical attributes (e.g., the thickness of the sensors).

Reference is now made to FIG. 11 which shows a nonlimiting example of the influence of accelerated degradation in the physical attributes of a sensor on the calculated mathematical correlation, according to some embodiments of the invention. Sensors having substantially the same chemical composition were heated to elevated temperature for 3, 6 and 17 hours in order to accelerate the thickness degradation of the sensors. The base resistance was measured after each heating cycle. After each heating cycle the sensors were exposed to a known VC to which the maximal subtracted resistance was extracted from signals received from the sensors. The graph shows the linear correlation between the maximal subtracted resistance and the base resistance of all tested sensors regardless to the deviation in production processes or the degradation processes of the sensors. Accordingly, a mathematical correlation calculated for sensors having the same chemical composting, may not change in the entire service time of all sensors fabricated at the same production line.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A volatile compounds (VCs) sensing device, comprising:
one or more scent recorders, each scent recorder comprising:
a plurality of sensors from which at least two have substantially the same chemical composition and differ in at least one known physical attribute;
a controller; and
electrodes for connecting the one or more scent recorders to the controller.

2. The VCs sensing device of claim 1, wherein the at least one known physical attribute is selected from: the sensor's thickness, the sensor's layer coverage, layer centering, layer morphology, the sensor's porosity, the sensor's tortuosity, the sensor's particles size, the sensor's particles distribution, thickness uniformity, organic ligands coating a conductive particle, electrode dimensions, a gap between electrodes, and a water contact angle of the sensors' surface.

3. The VCs sensing device of claim 1, wherein each sensor is composed of a plurality of conductive particles each being covered by organic ligands.

4. The VCs sensing device of claim 1, wherein the controller is configured to:
receive measured attribute values from of the at least two sensors;
receive signals from the at least two sensors, in response to exposure to at least one type of VC;
extract values of a feature from the signals; and
find a mathematical correlation between the extracted values and a corresponding measured attribute value.

5. The VCs sensing device of claim 4, wherein the received signals are from at least one known VC and the controller is further configured to:
associate the mathematical correlation with the at least one known VC and store it in a database.

6. The VCs sensing device of claim 5, wherein the received signals are from at least one unknown VC and the controller is further configured to:
identify the at least one unknown VC based on the stored mathematical correlation.

7. The VCs sensing device of claim 6, wherein identifying the at least one unknown VC comprises at least one of: identifying the type of the VC and the concentration of the VC.

8. The VCs sensing device of claim 1, wherein the measured attribute values are selected from: base resistance, base conductivity, electrical noise, base current, based voltage, base frequency, base amplitude

9. The VCs sensing device of claim 1, wherein the extracted feature values are selected from: the maximal subtracted resistance, the difference between maximum and minimum values, the average value, the maximum value, the minimum value, the first time derivative, the second time derivative, Signal to noise ratio, incline gradient, decline gradient, rise time, overshooting value relative to steady-state value, oscillation decay in time and oscillation frequency.

10. The VCs sensing device of claim 1, wherein the mathematical correlation is one of: a linear correlation, a parabolic correlation, a polynomial correlation, logarithmic correlation, exponential correlation, and power correlation.

11. A method of finding a mathematical correlation between extracted values and
corresponding measured attribute values, comprising:
receiving at least one measured attribute from at least two sensors having substantially the same chemical composition and differ in at least one physical attribute;
receiving signals from the at least two sensors, in response to exposure to at least one VC;
extracting values of a feature from at least some of the plurality of signals;
finding the mathematical correlation between the extracted values and corresponding measured attribute values.

12. The method of claim 11, wherein receiving signals is in response to exposure to at least one known VC and the method further includes:
associating the mathematical correlation with the at least one known VC and storing the correlation in a database.

13. The method of claim 12, further comprising:
receiving signals from the at least two sensors, in response to exposure to at least one unknown VC; and
identifying the at least one unknown VC based on the stored mathematical correlation.

14. The method of claim 12, further comprising:
training a machine learning (ML) module to identify the known VC based on the mathematical correlation.

15. The method of claim 14, further comprising:
receiving signals from the at least two sensors, in response to exposure to at least one unknown VC; and
identifying the at least one unknown VC based on the stored mathematical correlation, using the trained ML module.

16. The method according to claim 1, wherein the measured attribute values are selected from: base resistance (e.g., background resistance), base conductivity, electrical noise, base current, based voltage, base frequency, and base amplitude.

17. The method according to claim 1, wherein the extracted feature values are selected from: the maximal subtracted resistance, the difference between maximum and minimum values, the average value, the maximum value, the minimum value, the first time derivative, the second time derivative, Signal to noise ratio, incline gradient, decline gradient, rise time, overshooting value relative to steady-state value, oscillation decay in time and oscillation frequency.

18. The method according to claim 1, wherein the mathematical correlation is one of: a linear correlation, a parabolic correlation, a polynomial correlation, logarithmic correlation, exponential correlation, and power correlation.

19. The method according to claim 1, wherein the at least one physical attribute is selected from: the sensor's thickness, the sensor's layer coverage, the sensor's porosity, the sensor's tortuosity layer centering, layer morphology, the sensor's particles size, the sensor's particles distribution, thickness uniformity, organic ligands coating a conductive particle, electrode dimensions, a gap between electrodes and a water contact angle of the sensors' surface.

20. (canceled)

21. A method of calibrating sensors included in a scent recorder, comprising:
receiving measured attribute values from a plurality of sensors included in the scent recorder from which at least two sensors have substantially the same chemical composition and vary in at least one physical attribute, and wherein values of the at least one physical attribute are unknown;
receiving signals from the at least two sensors, in response to exposure to at least one type of VC;
extracting values of a feature from at least some of the signals;
finding a mathematical correlation between the extracted values and corresponding measured attribute values; and
calibrating values extracted from new signals received from the at least two sensors using the mathematical correlation.

22.-26. (canceled)
